(19) United States
(12) Plant Patent Application Publication
(10) Pub. No.: US 2002/0194655 P1
(43) Pub. Date: Dec. 19, 2002

(54) Raspberry variety named 'Motueka'

(76) Inventor: Harvey K. Hall, Motueka (NZ)

Correspondence Address: GREENLEE WINNER AND SULLIVAN P C, 5370 MANHATTAN CIRCLE, SUITE 201, BOULDER, CO, 80303, US

(21) Appl. No.: 09/866,445

(22) Filed: May 25, 2001

(51) Int. Cl. A01H 5/00

(52) U.S. Cl. PLT/204

(57) ABSTRACT

A new and distinct floricane fruiting variety of red raspberry, named ‘Motueka’, botanically identified as Rubus idaeus L. is described. The new variety is distinguished from others by its high yields of medium sized, high flavored, moderately bright and medium red berries. The plant exhibits a spine-free upright growth habit of medium vigor. The fruit are suitable for consumption as fresh berries and are also amenable to processing. Fruit are detached easily and the variety is well suited for harvest by machine as demonstrated in trials with a Korvan 9000 harvester. In addition, the plant has displayed resistance to Raspberry Bushy Dwarf Virus (RBDV).

### BACKGROUND TO THE INVENTION

The new variety of red raspberry, Rubus idaeus L, was created in the course of a planned breeding program carried out at HortResearch Nelson, New Zealand. The parents used to make the cross in 1989 were the selections B257 (seed parent) and F29 (pollen parent). B257 was selected from open pollinated seed of the Scottish Crop Research Institute selection 7936F5 grown at HortResearch Nelson. F29 was selected from the cross ‘Marcy’×‘Malling Delight’ produced on behalf of HortResearch at the Canada Agriculture Station at Abbotsford, British Columbia and grown at HortResearch Nelson.

The parentage of the new variety also includes the Scottish varieties ‘Glen Prosen’ (not patented) and ‘Glen Clova’ (not patented), the German variety ‘Rumiloba’ (not patented) and the United States variety ‘Carnival’ (not patented). The background of ‘Motueka’ also includes Rubus occidentalis via the Scottish breeding program and spinelessness from the old Scottish variety ‘Bumetholm’.

Seedlings were grown in the field at HortResearch Nelson and the original plant of the new variety was selected during the 1992-93 summer (Southern Hemisphere) and was found to exhibit:

(a) a spine-free upright growth habit of medium vigor,

(b) the ability to form attractive, medium sized mid red fruit of good flavor in exceptionally high yields on medium length fruiting laterals,

(c) resistance to Raspberry Bushy Dwarf Virus (RBDV), and

(d) adaptation for machine harvest.

The new variety was first asexually propagated in 1993, reproduced by root cuttings. The resulting plants propagated true to type demonstrating that the characteristics of the new variety are stable and are transmitted without change through succeeding generations.

### SUMMARY OF THE INVENTION

The new variety was tested and evaluated during the period 1995 to 2000 at HortResearch Nelson.

When compared to the parent B257, the new variety is found to form larger, almost as firm fruit, in higher yields. ‘Motueka’ is further distinguished from B257 by having fruit that are thicker, longer, more conical, with darker color, increased shininess, and reduced force required to separate the berry from the plug.

When compared to the F29 parent the new variety exhibits larger, coherent, non crumbly, medium red fruit in higher yields, a similar picking date (i.e. mid season), and a longer picking period. ‘Motueka’ is further distinguished from F29, by having no spines on juvenile or mature canes, by reduced cane vigor and cane number, and by having larger fruit that are very easy to separate from the plug.

Data collected during the evaluation period comparing fruiting performance of the new variety with standard New Zealand varieties is presented in Table 1.

TABLE 1
Comparison of fruiting performance.

| | Average Yield | Berry Weight |
|---|---|---|
| Variety | (T/ha) | (g) |
| ‘Motueka’ | 28.6 | 3.5 |
| ‘Marcy’ | 21.1 | 2.8 |
| ‘Skeena’ | 15.8 | 3.3 |
| ‘Chilliwack’ | 15.0 | 2.9 |

The data presented in Table 1. demonstrates the high fruit yield potential of the new variety. Berries of ‘Motueka’ are suitable for consumption as fresh fruit and are very well suited for processing. The color of the processed product is a similar red to that of ‘Marcy’ and ‘Skeena’, the standard varieties for processing in New Zealand, although lighter than that of ‘Meeker’ or ‘Willamette’, the standard varieties for processing in the United States.

### BRIEF DESCRIPTION OF THE DRAWINGS

The accompanying photographs show typical specimens of the new variety in color as true as is reasonably possible. The photographs were prepared during 1999 and 2000 and depict three year-old plants and plant parts grown outdoors at HortResearch Nelson, New Zealand

FIGURE 1. Illustrates a fruiting plant of the new variety showing the spineless growth and productive hanging fruiting laterals.

FIG. 2. Illustrates enlarged close-up end and side views of typical fruit of the new variety.

FIG. 3. Illustrates the shoot tip of a primocane of the new variety with the leaflets at various stages of development.

FIG. 4. Illustrates fully opened leaflets of the new variety from a floricane showing the upper and lower surface of the leaves.

FIG. 5. Illustrates the dormant canes of the new variety showing the light tan cane color and the complete absence of spines on the canes.

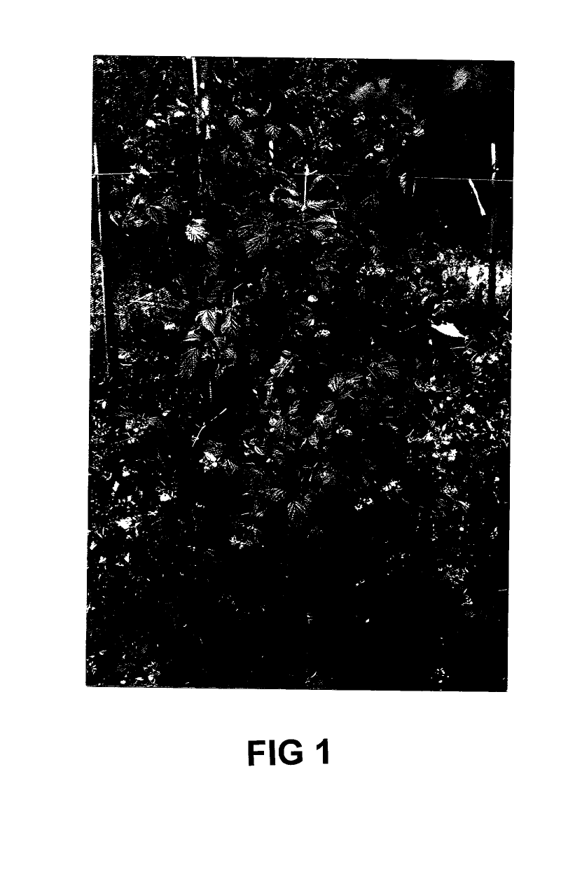

### DETAILED DESCRIPTION

The specimens described were three years old growing in the field at HortResearch Nelson. The observations were made in the 1999-2000 season.

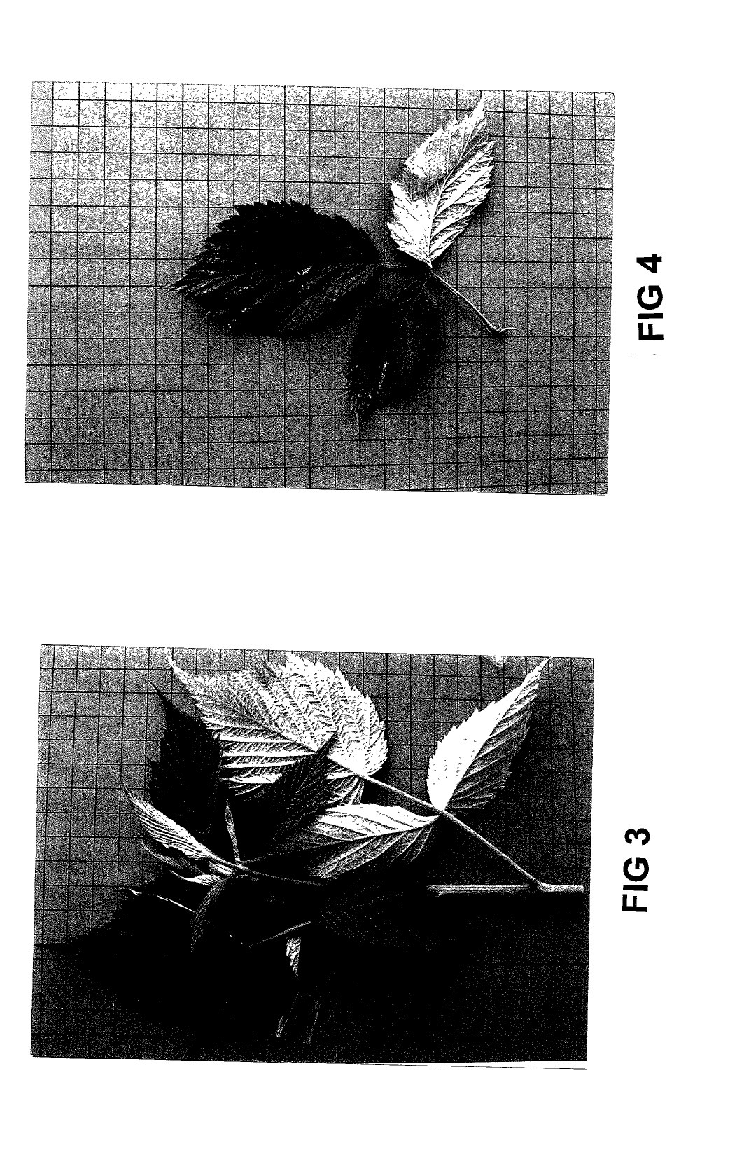

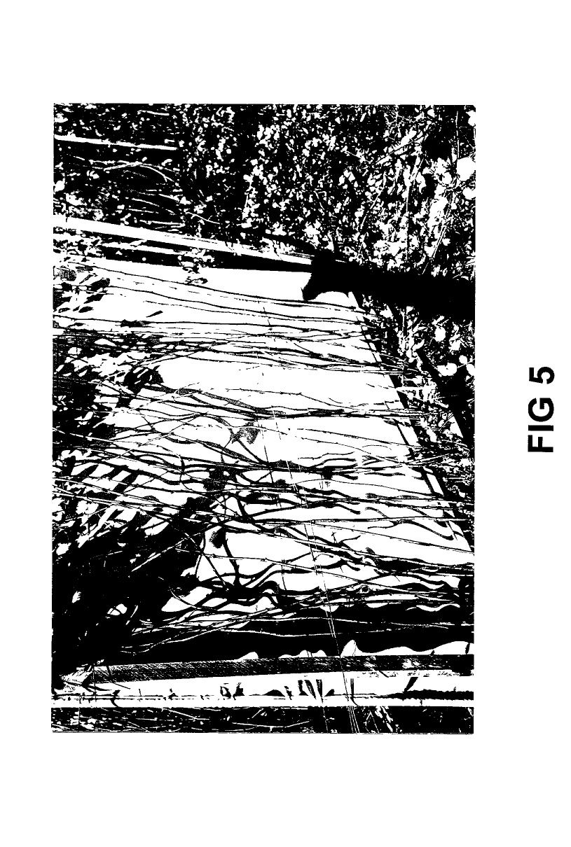

Plant and foliage The plant exhibits a very upright growth habit (FIG. 5). Typical mature plant height is commonly in the range 175 to 195 cm, although may vary with the growing conditions. Moderate vigor is exhibited. Spines are absent on new canes, and also on those developed in the previous season. The canes are upright with a leafy presentation, and are typically a light brown-tan coloration during the winter. The fruit is borne primarily on the previous year's growth. The leafy coverage tends to provide hand pickers with poor fruit presentation at harvest time but the leafiness promotes effective harvest by machine. The leaves are moderately crinkled, flat, and moderately glossy (FIGS. 3 and 4). New shoots commonly show weak anthocyanin coloration. The number of leaflets per internode is predominantly three.

Inflorescence White flowers are borne on short slender pedicels that lack spines. The time of bloom is mid-season for a summer-fruiting raspberry. The flowers have five petals with the pedicel length typically in the range 2 to 3 cm. However, the more basal the pedicel the longer it commonly becomes with pedicel lengths up to about 5 cm being observed. A typical flower diameter is approximately 2.2 cm. The flowers are predominantly borne singly, although sometimes in clusters of two or more. Terminal branch flower clusters frequently consist of two flowers and basal flower clusters may number three to five. The flowers have no discernible fragrance.

Harvest At HortResearch Nelson, the typical start date for picking the new variety is December 8. Fifty 50 percent of the harvest is typically completed by December 27, and harvest ceases approximately January 28. The harvest period is commonly longer for ‘Motueka’ than for either ‘Marcy’ or ‘Skeena’. Similarly, the date at which 50 percent of harvest is complete is approximately 10 days later for ‘Motueka’ than for ‘Marcy’ and 4 days later than that for ‘Skeena’. ‘Motueka’ is well suited for harvest by machine. In trials with a Korvan 9000 harvester a high percentage of ripe fruit was removed and successive harvests were uniform and high quality. Few green fruit were removed, even at high beater frequencies and the amount of reject fruit to be removed from grading belts was minimal.

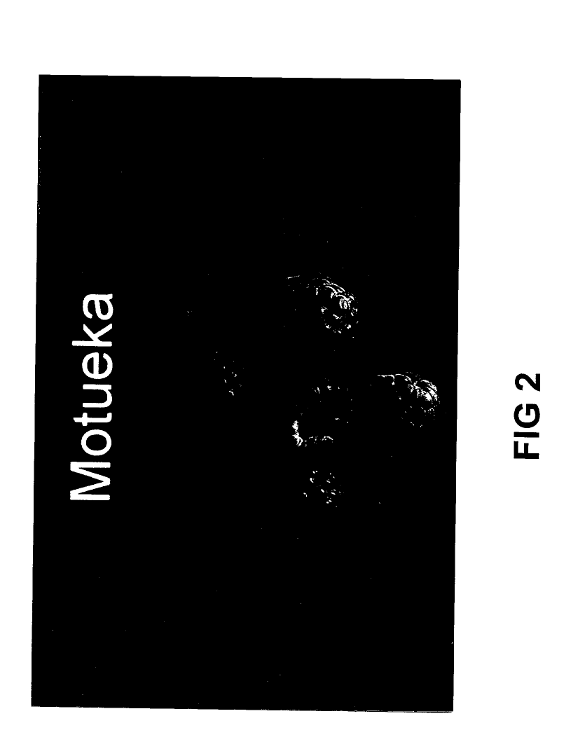

Fruit The berries formed on ‘Motueka’ are medium-large in size. The fruit is short conical in configuration (FIG. 2) and is bright in appearance with medium glossiness. The berries are medium-firm and fleshy, with good flavor. Berries generally weigh approximately 3.5 g, although larger fruit, up to 5 g, may be observed. Fruit of ‘Motueka’ are well suited for individually quick frozen storage and excellent for processing, producing a high flavored product.

Pest and disease resistance Resistance to aphids is unknown. Since the selection of ‘Motueka’ in 1993 numerous tests for raspberry bushy dwarf virus (RBDV) have been done using ELISA but on no occasion has the virus been detected despite high infection pressure. From this we suggest that ‘Motueka’ is likely to be resistant to the common strain of RBDV found in New Zealand. If ‘Motueka’ is grown in dense rows and conditions are favorable for the infection of gray mould (Botrytis cinerea) some problems with fruit rot may be encountered. The susceptibility to fruit rot has been observed to be less than that shown by ‘Skeena’ or ‘Marcy’.

1. A new and distinct variety of raspberry plant, rubus idaeus l, substantially as herein shown and described.
